(19) United States
(12) Patent Application Publication
(10) Pub. No.: US 2005/0222258 A1
(43) Pub. Date: Oct. 6, 2005

(54) Pharmaceuticals comprising shikonins as active constituent

(76) Inventor: Feixin Wang, Beijing (CN)

Correspondence Address: YI LI, CUSPA TECHNOLOGY LAW ASSOCIATES, 11820 SW 107 AVENUE, MIAMI, FL, 33176, US

(21) Appl. No.: 11/129,255

(22) Filed: May 14, 2005

Related U.S. Application Data
(63) Continuation-in-part of application No. PCT/CN03/00138, filed on Feb. 21, 2003.

(51) Int. Cl. A61K 31/22

(52) U.S. Cl. 514/546; 514/549

(57) ABSTRACT

The object of the invention is to provide medicaments containing Shikonin (include shikonin and alkannin) compounds and salts thereof, which are used for treatment or prevention of microorganism infection in human body, inflammation, malignant tumor, hemorrhage, hematopathy, and autoimmune disease.

### CROSS REFERENCE TO RELATED APPLICATION

This application is a Continuation-in-part application of PCT patent application No. PCT/CN2003/000138 filed Feb. 21, 2003, which is hereby incorporated by reference in its entirety.

### TECHNICAL FIELD

The present invention is involved in a medicament containing Shikonin compounds or its salt, (include shikonin and alkannin), specially the medicament containing Shikonin compounds or its salt a an active component designing to prevent and treat microorganism infection in human body, inflammation, tumour, hemorrhage, hematopathy, SARS disease and autoimmune disease.

### TECHNICAL BACKGROUND

Shikonin compounds are an opened compound reported in literatures (Lin Zhibin, et al, P101-105, Issue 2, Volume 12, 1980, JOURNAL OF BEIJING MEDICAL UNIVERSITY), therein, the Shikonin compounds have the following general formula structure:

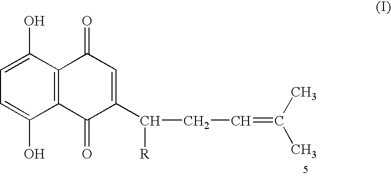

Shikonin compounds are insoluble in water but freely soluble in oil, alcohol or ethers, abstracted from Boraginaceae plants: lithospermum erythrorhizo Sieb.et zucc.; Arnebia euchroma (Royle) Johnst. It's known that the Zicao mixture extract has some functions such as anti-inflammation, but it's just mixed in the form of mixture extract when medicament delivery. While it has not been reported yet that the Zicao quinone compound extracted from plant Shikonin and artificial or biosynthetic Shikonin quinone compounds are designed to manufacture medicaments in single compound or combination of several compounds, particularly for prevention and treatment of microorganism infection in human body, inflammation, tumour, hemorrhage, hematopathy and autoimmune disease.

### CONTENT OF INVENTION

Therefore, the present invention is designed to provide a single compound or several compounds separated from Zicao extract to manufacture a medicament for prevention and treatment of microorganism infection in human body, inflammation, tumor, hemorrhage, hematopathy and autoimmune disease.

The present invention provides a medicament to prevent and treat microorganism infection in human body, inflammation, tumour, hemorrhage, hematopathy, and autoimmune, which comprises one to five Shikonin compounds or its salt represented in the following Formula (1) 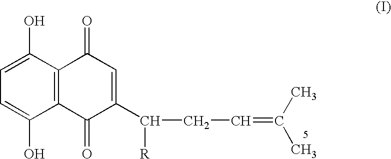
wherein, R is a group selected from a group composed of H (deoxyshikonin), OH(Shikonin), (CH₃)₂C═CHC(O)O-(β,β-dimethylacry), CH₃C(O)O-(Acetylshikonin), (CH₃)₂C═C(CH₃)CH₂C(O)O-(teracrylshikonin), (CH₃)₂COHCH₂C(O)-(β-hydroxyisovalerylshikonin), (CH₃)₂C[OC(O)CH₃]CH₂C(O)O-(β-acetoxyisovalerylshikonin); and preferably, said medicament contains 1 to 3 compounds selected from Shikonin, β,β-dimethylacryishikonin and Acetylshikonin; more preferably, said medicament contains β,β-dimethylacrylshikonin and/or Acetylshikonin; the most preferably, said medicament contains β,β-dimethylacryishikonin. The salts of Shikonin compounds in this invention include the salts of alkali metals, alkaline earth metal and ammonium, etc thereof.

The medicament according to this invention contains one or more compound(s) of the Shikonin compounds as raw medicaments, of which the purity of single compound is 80% or more, and the perferable purity is 90% or more. When the meicine contains a combination of several compounds, the effective components thereof is 70% or more.

If necessary, the invented medicament can further contain other active components. There is no extraordinary restriction to the other active components, which the technologist can select properly in accordance to the existing technology.

The content scope of Shikonin compounds in the invented medicament ranges from 0.0001% to 75% (weight percent), which can be selected properly according to different preparation as well as symptoms of disease. When being used in human body, the daily consumption of the mentioned Shikonin compounds can be controlled between 10 μg-20 g, the perferable one is 10 μg-10 g, and the more perferable one is 1 mg-8 g, and the best one is 5 g, which can be selected properly in accordance to the different status such as age, weight and state of illness for different sufferers. It can be used for a single time or several times. The invented medicament can be delivered in oral administration, external application, injection, inhalation or skin penetration.

The Shikonin compounds in the invention can be used for prevention and treatment of microorganism infection including pathogenic Gram-positive micrococcus, such as staphylococcus, streptococcus pneumonia, staphylococcus epidermidis and enterococcus; pathogenic Gram-negative micrococcus such as Klebsiella pneumonia ozaenae, Serratia marcesens, Stenotrophomonas maltophilia; anaerobic or little aerobic pathogen such as Helicobacter pylori; Eumycetes such as deep and superficial eumycetes; Leuconostoc spp, aspergillus fumigatus, cryptococcus, dermatophyte, krusei leuconostoc spp, Cercospora punicae, etc; and all kinds of mycoplasma infection particularly the mycoplasma infection of the respiratory system; virus such as hepatitis B virus, cold virus, herpes virus and HIV virus, etc. SARS virus such as coronavirus and its variation etc.

The Shikonin compounds can be used for prevention and treament of inflammation of human body, including phlebitis, vascular purpura, colpitis and edema, etc.

It also can be used for prevention and treatment of hemorrhage and hematopathy in human body, for instance, burning, scalding, all kinds of dermatitis, serticemia hemophilia, primary thrombocythemia, leukaemia, etc.

It also can be used for prevention and treatment of tumour especially malignant tumor, for instance, ascitic type tumour: liver cancer, L1210; solid tumour: W256, S180, gastric cancer 823, squamous cell carcinoma 109, Lewis lung cancer, etc.

The medicament containing Shikonin compounds in the present invention can be used for prevention and treatment of autoimmune disease of human body, i.e. promoting human body's functions of nonspecific immunity and idiosyncratic cell-mediated immunity through improving the function of immune response of T lymphocytes.

Therefore, the medicament according to the present invention are available for respiratory system, digestive system, urinary system, reproductive system, blood system, circulating system and skin or mucous membrane in human body.

### Mode Of Carrying Out The Invention

The following text gives detailed description on the manufacture of pharmaceutical preparation containing Shikonin ompounds and pharmacodynamics experiments of the present invention, but the protection scope of the present invention is not limited to this.

### PREPARATION EXAMPLE 1

Shatter 2 kg Arnebia euchroma (Royle) Johnst. components, make abstraction with petroleum ether till residue of Arnebia euchroma (Royle) Johnst is colorless, recover the solvent and get 80 g dark red paste. Separate the paste through silica gel H-column liquid chromatography and carry out gradient elution with 1%-20% ethyl acetate-petroleum ether, and then get 7 monomers of Shikonin compounds stated above, i.e. 2.944 g deoxyshikonin (yield is 3.68%), 0.712 g Shikonin (yield is 0.89%), 29.024 g β,β-dimethylacrylShikonin (yield is 36.28%), 13.27 g Acetylshikonin (yield is 16.59%), 6.032 g teracrylshikonin (yield is 7.54%), 0.776 g β-hydroxyisovaleryshikonin (yield is 0.97%), 0.792 g β-acetoxyisovalerylshikonin (yield is 0.99%). By high-pressure liquid chromatography, all purity is over 90%.

### PREPARATION EXAMPLE 2

Shatter 2 kg Arnebia euchroma (Royle) Johnst. components, go through 20-40 meshes and get 70 g red ointment by CO₂-supercritical extraction. Separate the cream by high-pressure liquid preparative chromatography (Germany Knauer K1001 type) with preparative column: silica gel H 10 μm 50×300 mm and carry out gradient elution with 1%-20% ethyl acetate-petroleum ether, and then get 7 red monomers of Shikonin compounds stated above, i.e. 3.486 g deoxyshikonin (yield is 4.98%), 0.707 g Shikonin (yield is 1.01%), 30.877 g β,β-dimethylacrylshikonin (yield is 44.11%), 15.869 g Acetylshikonin (yield is 22.67%), 6.034 g teracrylshikonin (yield is 8.62%), 0.91 g β-hydroxyisovalerylshikonin (yield is 1.30%) and 0.77 g β-acetoxyisovalerylshikonin (yield is 1.10%). By high-pressure liquid chromatography, all purity is over 90%.

### EXAMPLE 1

Manufacture troche with single or several combination of the above 7 compounds according to the way widely known by technical personnel of the field, of which the troche with 10%-70% Shikonin compounds can be made according to actual demand.

Take 100 g β,β-dimethylacrylshikonin got in Manufacture example 1 or Manufacture example 2, 100 g nucleated fiber, 30 g magnesium stearate, and 4 g hydroxypropyl methyl cellulose under the aseptic operation conditions. 0.5 g tallet can be made according to widely known troche made technology and equipment.

### EXAMPLE 2

Manufacture 0.5 g tallet with 100 g combination of Shikonin componds got in Manufacture example 1 or Manufacture example 2 (combination proportion of Shikonin, β,β-dimethylacrylshikonin and Acetylshikonin is 1:1:2) and the left in the same way as Implementation example 1.

### EXAMPLE 3

Manufacture the ointment of the above 7 Shikonin compounds according to the way widely known by technical personnel of the field, of which the ointment with 0.0001%-10% Shikonin compounds can be made according to actual demand. Under the aseptic operation conditions, take 0.5 g Shikonin compounds got in Manufacture example 1 or Manufacture example 2 (combination proportion of deoxyshikonin, Shikonin, β,β-dimethylacrylshikonin Acetylshikonin and β-hydroxyisovalerylshikoninis 0.7:1:1:2:0.5), 80 g vaseline, 10 g liquid paraffin and 10 g anhydrous lanolin and equably triturate them into products in separate bags for external use. This ointment also can be made into patch for skin penetration in a way widely known by technical personnel of the field.

### EXAMPLE 4

Manufacture the injection of the above 7 Shikonin compounds according to the way widely known by technical personnel of the field. Under the aseptic operation conditions, take 0.5 g β,β-dimethylacrylshikonin got in Manufacture example 1 or Manufacture example 2, 400 ml propylene glycol, 100 ml ethanol, 20 ml tween-80 and 15 ml benzyl alcohol, make them fully dissolved and add water up to 1,000 ml. After mixing well, bottle them to be injection product.

The following description is on test result of effect of the medicament containing Shikonin compounds.

(1) Dispensation of the Drug

Respectively take 5.0 mg Shikonin, β,β-dimethylacrylshikonin, Acetylshikonin got in Manufacture example 1 or Manufacture example 2. Make the medicament dissolved in 1 ml DMSO. After diluting by 50 times with RPMI-1640 culture medium, separately pack them and further dilute into the following concentration: 100, 50, 25, 12.5, 6.25, 3.125, 1.5625, 0.78125, 0.390625 (μg/m).

(2) Sensitivity Test of the Drug

Separately pack the medicament at above concentration into the orifice plate and vaccinate with all bacterial strains at a density of 10³-10⁶.

The test result indicates that Shikonin, β,β-dimethylacrylshikonin and Acetylshikonin have high sensitivity to Gram-positive staphylococcus aureus and the MIC is 0.391-12.5 μg/ml; for Gram-negative pathogen, the MIC of pneumobacillus is 0.391-6.25 μg/ml and that of some bacterial strains is 12.5-50 μg/ml; most isolates of bacillus prodigiosus and most bacterial strains of stenotrophomonas bacilli have a MIC of 0.391-3.125 μg/ml. Therein, they are especially effective to stenotrophomonas bacilli and the MIC is 0.391-0.781 μg/ml while that to berberine is 8-32 μg/ml, i.e. it is obviously better than berberine. For bacteroid, especially bacteroides fragilis, the MIC is 0.391-6.25 μg/ml; they are highly sensitive to Helicobacter pylori and the MIC is 0.391-0.781 μg/ml.

Additionally, the result of β,β-dimethylacrylshikonin invitro antifungal test indicates that the MIC for candida and cryptococcus is 2.08-33.3 μg/ml and MIC₉₀ is 33.3 μg/ml; for fluconazole the MIC is 0.125-64 μg/ml and MIC₉₀ is 69 μg/ml; to dermatophyte the MIC is 4.16-8.32 μg/ml with MIC₉₀ of 4.16 μg/ml while the MIC of fluconazole to most bacterial strains of dermatophyte is 32-64 μg/ml with MIC₉₀ of 64 μg/ml. There are obvious differences in both of them. Furthermore, β,β-dimethylacrylshikonin has good inhibitory effect to C. krusei that resists fluconazole and the MIC is 8.32-16.6 μg/ml, and for Pseudallescheria boydii that is insensitive to most antifungal medicaments like fluconazole, the MIC is 4.16-8.32 μg/ml. Besides, the MIC of Acetylshikonin against cryptococcus neoformans is 3.90625 μg/ml, against red trichophyton is 0.90625-62.5 μg/ml; the MIC of β,β-dimethylacrylshikonin against aspergillus fumigatus, cryptococcus and red trichophyton is 3.0625-25011 g/ml. Therefore, Shikonin compounds are broadspectrum and effective antifungal drug.

In addition, Shikonin compounds of the invention have a MIC of over 200 μg/ml on the microbes like Lactobacilli and Bifidobactirium beneficial for human body. Therefore, the above data indicates that medicaments with Shikonin compounds in the invention are sensitive to pathogenic microorganism but insensitive to microbes beneficial for human body.

From the comparative experiment between the mixed extraction from Zicao and 1-3 kinds of Shikonin compounds, it is observed that the medicaments containing Shikonin compounds are obviously better than mixed extraction from Zicao; the results of MIC (μg/ml) are shown in Table 1.

TABLE 1

| Name of bacterial strain | A | B | C |
|---|---|---|---|
| Staphylococcus epidermidis | 12.5 | 0.391 | 0.7812 |
| Serratia marcescens | 25 | 0.781 | 3.125 |
| Bacteroid | >200 | 0.391 | 0.391 |
| Candida albicans | 500 | 3.9062 | 250 |

Note:
A is mixed extraction from Zicao
B is β,β-dimethylacrylshikonin;
C is mixture of Shikonin compounds (the mixture ratio of Shikonin, β,β-dimethylacrylshikonin and Acetylshikonin is 1:1:2)

The experiment of Shikonin, β,β-dimethylacrylshikonin and Acetylshikonin's bacteriostatic effect on mycoplasma pneumoniae shows that, their MIC for mycoplasma pneumoniae are respectively 3.751 μg/ml, 2 μg/ml and 7.819 μg/ml, equivalent to the inhibitory effect of 0.1925 μg/ml erythrocin.

The following table shows the test results for using Shikonin compound ointment made in Implementation Example 3 as external remedy for treating some disease.

TABLE 2

| | Number of | Effective | Cured | Days of | Medicament | |
|---|---|---|---|---|---|---|
| Cases | subjects | percentage | percentage | treatment | Delivery route | Note |
| Burn & scalding | 300 | 100% | 100% | 6-20 | Direct delivery | 92 people scalded, 186 |
| | | | | | at affected part | second degree superficial |
| | | | | | | burns, 114 deep second degree |
| | | | | | | and third degree burns |
| Hemorrhoids | 117 | 100% | 97.4% | 15 | Direct delivery | Recurrence in three cases |
| | | | | | at affected part | after half a year |
| Herpes zoster | 98 | 100% | 100% | 3-7 | Direct delivery | Polyinosinic-polytidylin |
| | | | | | at affected part | acid is used in 12 cases |
| Cervical erosion | 80 | 100% | 100% | 10-20 | Vagina delivery | |
| Children's nosebleed | 257 | 99.6% | 72.8% | 15 | Nasal cavity delivery | |
| Verruca plana | 100 | 96% | 81% | 10-30 | Direct delivery | |
| | | | | | at affected part | |
| Chronic prostatitis | 40 | 82.5% | 57.5% | 10-20 | Anus delivery | |
| Acne | 50 | 92% | 60% | 15 | Direct delivery | |
| | | | | | at affected part | |
| Bedsore | 30 | 100% | 100% | 7-21 | Direct delivery | |
| | | | | | at affected part | |
| Eczema rhagadiforme | 98 | 94.9% | 66.3% | 10-30 | Direct delivery | |
| | | | | | at affected part | |
| Verruca acuminata | 55 | 100% | 100% | 5-35 | Direct delivery | |
| | | | | | at affected part | |
| Infantal diaper | 208 | 100% | 100% | 2-6 | Direct delivery | |
| dermatitis | | | | | at affected part | |

It is observed from the above table that the external remedy of Shikonin compounds is suitable for treatment of most abscess, wound, scabies and herpes; the effect is prominent for burn and scalding without cicatrices after recovery.

Table 3 shows the animal test results for using Shikonin, β,β-dimethylacrylshikonin and Acetylshikonin to restrain tumor.

TABLE 3

| | β,β- | | | | | |
|---|---|---|---|---|---|---|
| | dimethylacrylshikonin | | Acetylshikonin | | Shikonin | |
| | Tumor | Life | Tumor | Life | Tumor | Life |
| Type of | inhibitory | prolonged | inhibitory | prolonged | inhibitory | prolonged |
| tumour | rate | rate | rate | rate | rate | rate |
| Ascitic tyre | | 113.4% | 47.8% | 112.6% | | 130.8% |
| liver cancer | | | | | | |
| S180 | 9.63% | | 35.7% | | | |
| Lewis lung | 42.8% | | 52.6% | | | |
| cancer | | | | | | |
| L1210 | | | | 128% | | |
| W256 | | | 77% | | | |

It is observed from the above table that β,β-dimethylacrylshikonin has different extent of therapeutic effect for liver cancer, S180 and Lewis lung cancer; Acetylshikonin has different extent of therapeutic effect for liver cancer, S180, L1210 and Lewis lung cancer and W256; Shikonin is only effective for liver cancer. As is shown in the experiment using Shikonin compounds to restrain virus, oral dosing β,β-dimethylacrylshikonin is made on the 7th day since duck is infected by DHBV with 100 mg/kg and twice a day, the inhibitory effect for DHBV-DNA level in blood serum of infected duck is prominent in 10 days (P<0.05-0.01) without toxic reaction; for the 50 mg/kg group, significant inhibitory effect is shown (P<0.05). As is shown in the experiment using β,β-dimethylacrylshikonin to restrain HBV, if concentration is 30 μg/ml, average inhibitory rate for HBsAg is 96.2601% and for HBeAg is 91.6056%. Table 4 shows the invitro test results of Shikonin and β,β-dimethylacrylshikonin resisting HIV-1 reverse transcriptase and integrase.

TABLE 4

| | | IC₅₀ (μg/ml) |
|---|---|---|
| Resisting HIV reverse | Positive control PFA | 0.097 |
| transcriptase | Shikonin compounds | >20 |
| Resisting HIV integrase | Positive control ABPS-Y | 0.922 |
| | Shikonin compounds | 12.467 |

Study the effect of Shikonin compounds with the model of mouse's low immunologic function caused by mitomycin C. If 6 mg/kg β,β-dimethylacrylshikonin is injected in the abdominal cavity for 5 days without interruption, the cell toxicant in the mouse's splenic cell and NK cell increases by around 20% (P<0.001), which indicates that β,β-dimethylacrylshikonin can recover the injury of intraperitoneal macrophage, improve the migration ability of intraperitoneal macrophage, raise the activity of T lymphocytes, and promote the immune response of T lymphocytes, enhance the nonspecific immunity and specific cell immunity effect of body.

Experimental design for the effect of Shikonin salt to SARS virus.

1) Objects Selection

Definite diagnosed SARS patients when admited in hospital with swallowing allility and seveve symptom, within them 20 patients with similar conditions were selected and divided into 2 groups, 10 patients in each group, one was test group the other was control group

2) Theraputic Plan

Test group: Recived hospital former therapeutic plan+“antitoxic capsule” 2 capsule Tid after meal oral administration.
Control group: Only tieated with hospital former therapeutic plan.
Treating period: 10 days.

Note: One antitoxic capsule contains Acetylshikonin 50 mg and subsidiay meterials (β-cycloheptan)250 mg, sach antitoxic capsule is 300 mg.

TABLE 5
Result of treatment

| | |
|---|---|
| Significant effect | effect |

| Test group | No, of patient | % | No, of patient | % | No effect |
|---|---|---|---|---|---|
| Major | 9 | 90 | 1 | 10 | 0 |
| parameter | | | | | |
| Subordinate | 10 | 100 | 0 | 0 | 0 |
| parameter | | | | | |

| Contol | significant | | effect | | No effect | |
|---|---|---|---|---|---|---|
| group | No, of patient | % | No, of patient | % | No, of patient | % |
| Major | 1 | 10 | 8 | 80 | 1 | 10 |
| parameter | | | | | | |
| Subordinate | 3 | 30 | 6 | 60 | 1 | 10 |
| parameter | | | | | | |

Major parameter: 1-2 weeks quicker rcoverd than control group.
There are three conditions as following.
(1) normal temperature for 7 days above.
(2) Respinatoly system symptoms tvnming better evidently.
(3) Shadow of chest x-ray was notably alsorfed
   Subordinate Parameter:

Medical image necovery speed faster than control group 7-10 days according to chest x-ray film.

From this experiment can be seen the nesults of using antiloxix capsule for SARS patients in early stage, the effect was significantly better than in late stage. the effect of using antitoxic capsule within one week after getting SARS is the best. Lately tieated with antitoxic capsule is better than not used this medicament, from the image of x-ray film can be clearly seen the infectious satuation of lung in SARS patients, Though the patients were living in hospital neveired tveatment but there were 5 patients' symptoms developed in contid group and there was one patient's symptoms developed in test group but not severe.

According to this analysis inhilitory effect of this medicament may kill virus not only can velease SARS havm to patients but at the same time also may Inhance the vstaration function of human body antitoxic capsule can inhifit and kill eamyetes, so this medicament may effectively parent secondary infections caused by eumycetes.

From the figures of the comparision of test group and contend group can be identified this point.

1. A medicament for prevention or treatment of microorganism infection in human body, inflammation, malignant tumor, hemorrhage, hematopathy SARS disease. and autoimmune disease, wherein the medicament contains 1 to 5 of Shikonin compounds and its salt shown in formula (1), 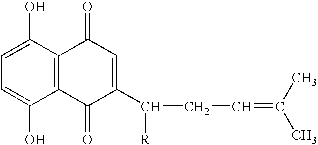
Wherein R is a group selected from H, OH, (CH₃)₂C═CHC(O)O—, CH₃C(O)O—, (CH₃)₂C═C(CH₃)CH₂C(O)O—, (CH₃)₂ COHCH₂C(O)O— and (CH₃)₂C[OC(O)CH₃]CH₂C(O)O—.

2. The medicament as stated in claim 1, wherein R is 1 to 3 groups selected from OH, (CH₃)₂C═CHC(O)O— and CH₃C(O)O—.

3. The medicament as stated in claim 1, wherein R is (CH₃)₂C═CHC(O)O— and/or CH₃C(O)O—.

4. The medicament as stated in claim 3, wherein R is (CH₃)₂C═CHC(O)O—.

5. The medicament as stated claim 1, wherein the purity of each compound is 80% or more.

6. The medicament as stated in claim 1, wherein the purity of each compound is 90% or more.

7. The medicament as stated in claim 1, wherein the effective components are 70% or more when the medicament contains more than one compound.

8. The medicament as stated in claim 1, wherein the medicament further contains other active components.

9. The medicament as stated in claim 1, wherein the medicament is used for treatment or prevention of microogomism infections of every system of human body which include stuptocowus pneumonia kililsiella hiicotacter Rylari candia cryptococcus dermatophyte every kind of mycoplarma infoctions include mycoplarma pneumonia: every kind of chlamgdia infection or virus infection include hepalities virus. Influenga virus, herpes virus and HIV and coronaviws and its variated virus caused SARS disease's pathogenic organism.

10. The medicament as stated in claim 1, wherein the medicament is used for treatment or prevention of cancers associated with hydroperitoneum tumour such as liver cancer and L1210, and entity tumor such as sarcoma 180, stomach cancer 823, squama carcinoma 109 or lung cancer.
